(19) United States
(12) Patent Application Publication
(10) Pub. No.: US 2019/0125843 A1
(43) Pub. Date: May 2, 2019

(54) Use of antiplatelet thrombolysin in the preparation of medicine for treating thrombotic thrombocytopenic purpura

(71) Applicant: Zhaoke Pharmaceutical (HEFEI) Company Limited, Anhui Province (CN)

(72) Inventors: Xiaoyi Li, Shatin NT (HK); Xiangrong Dai, Anhui Province (CN)

(73) Assignee: Zhaoke Pharmaceutical (HEFEI) Company Limited, Anhui Province (CN)

(21) Appl. No.: 16/095,607

(22) PCT Filed: Apr. 22, 2016
(86) PCT No.: PCT/CN2016/079973

§ 371 Date: Oct. 22, 2018

(30) Foreign Application Priority Data
Mar. 23, 2016 (CN) 201610180263.4

(51) Int. Cl. A61K 38/48; A61P 7/06; A61P 7/04; A61P 7/02

(52) U.S. Cl. CPC A61K 38/48; A61P 7/02; A61P 7/04; A61P 7/06

(57) ABSTRACT

A method and pharmceutical compositon for the treatment of thrombotic thrombocytopenic purpura. The method and pharmceutical compositon uses a therapuetically effective amount of an antiplatelet thrombolysin comprising two polypeptide chains, an alpha chain and a beta chain. The antiplatelet thrombolysin of the invention can limit platelet adhesion and aggregation, restore platelets, red blood cell and hemoglobin levels, at the same time, reduce the level of lactate dehydrogenase, and effectively inhibits thrombocytopenia and schistocyte hemolytic anemia, so as to achieve the purpose of the treatment of TTP, which has a good prospect of clinical use.

This application claims the priority of Chinese patent application No. 201610180263.4, named “use of antiplatelet thrombolysin in the preparation of a medicament for treatment of thrombotic thrombocytopenic purpura”, filed with Chinese Patent Office on Mar. 23, 2016, and the whole content thereof is incorporated into this application by reference.

### TECHNICAL FIELD

The invention relates to the field of medicine, in particular, to the use of an antiplatelet thrombolysin in the preparation of a medicament for treating Thrombotic Thrombocytopenic Purpura (TTP).

### BACKGROUND OF THE INVENTION

Thrombotic Thrombocytopenic Purpura (TTP) is a severe diffuse thrombotic microangiopathy, which is characterized by microvascular hemolytic anemia, consumptive reduction of platelet aggregation, and organ damage caused by micro thrombus development (such as kidney, central nervous system, etc.). The pathogenesis thereof is closely related to a vWF protein cleaving enzyme (von Willebrand factor, ADAMTS13). The role of the enzyme is to specifically cleave the von Willebrand factor (vWF) in the blood, so as to ensure the normal hemostatic effect of human body. In the course of TTP, due to a decrease in the activity of the enzyme, vWF is aggregated on vascular endothelial cells and become multiple super large polymers, which in turn can bind to the GPIb receptor on the surface of platelets in the blood stream, mediating adhesion and aggregation of platelet on endothelial cells, finally resulting in thrombosis and further development of TTP.

According to the etiology, TTP can be divided into hereditary TTP and acquired TTP. The difference between the two mechanisms is the different causes of ADAMTS13 enzyme activity decrease. Hereditary patients is due to lack of expression of this enzyme which is in congenital, which is caused by genetic defects. Among acquired TTP patients, the cause of reduced activity is that the patient can produce a kind of auto-antibodies directed against ADAMTS13, which results in enzyme inactivation.

For the above reasons, the most effective treatment for TTP is fresh plasma infusion and plasma exchange, but the drawback thereof is that it relys on objective conditions, and has a very high recurrence rate and mortality of TTP patients. For patients with acquired TTP, immunosuppressive drugs or immunoglobulin etc. can be used to inhibit the production of anti ADAMTS13 auto-antibody, however, this treatment takes effect slowly, the effect may vary from patient to patient, and is not exact. The supplement of ADAMTS13 protein is only effective for congenital TTP patients, because for the acquired TTP patients, the supplemented ADAMTS13 protein will soon be inhibited by auto-antibodies. In patients with TTP, 70%-80% are acquired TTP, therefore, the clinical use of this method is very limited.

Contents of the Invention In view of this, the technical problem to be solved by the invention is to provide use of antiplatelet thrombolysin in the preparation of a medicament for treatment of thrombotic thrombocytopenic purpura. It was found in the invention that antiplatelet thrombolysin in blood of TTP patients can restore blood platelet, red blood cell and hemoglobin level, at the same time, reduce the level of lactate dehydrogenase, effectively suppress thrombocytopenia and schistocyte hemolytic anemia, inhibit platelet aggregation, so as to prevent thrombosis and improve the symptoms of TTP.

The antiplatelet thrombolysin (APT) described in the invention is a proteolytic enzyme isolated from Agkistrodon acutus venom, consisting of two polypeptide chains: an alpha chain and a beta chain, wherein the amino acid sequence of the alpha chain is shown as SEQ ID NO.1, and the amino acid sequence of the β chain is shown as SEQ ID NO.2, which is disclosed in patent application 201310228219.2.

Preferably, thrombotic thrombocytopenic purpura is congenital or acquired thrombotic thrombocytopenia purpura.

Preferably, the medicament for treatment of thrombotic thrombocytopenic purpura provided by the invention is a chemical or biological preparation.

Preferably, the medicament for treatment of thrombotic thrombocytopenic purpura comprises antiplatelet thrombolysin and a pharmaceutically acceptable excipient.

Preferably, the medicament for treatment of thrombotic thrombocytopenic purpura provided by the invention is an oral preparation or injection.

More preferably, the oral preparation for the treatment of thrombotic thrombocytopenic purpura is in the form of tablet, capsule, pill, granule, drop pill, microcapsule or pellet.

More preferably, the mass-volume concentration of antiplatelet thrombolysin in the oral preparation or injection for the treatment of thrombotic thrombocytopenic purpura is 1.8-10 μg/mL.

More preferably, the dose of antiplatelet thrombolysin in the oral preparation or injection for the treatment of thrombotic thrombocytopenic purpura is 0.03-3 mg/kg.

The invention provides use of antiplatelet thrombolysin in the preparation of a medicament for the treatment of thrombotic thrombocytopenic purpura.

Antiplatelet thrombolysin can prevent and treat the main symptoms of acquired TTP in baboon model, restore platelet, red blood cell and hemoglobin levels, at the same time, reduce the level of lactate dehydrogenase, effectively suppress the thrombocytopenia and schistocyte hemolytic anemia, so as to achieve the purpose of treatment of TTP. Therefore, the use of the antiplatelet thrombolysin provided by the invention is different from the use disclosed in the prior art, and the mechanism thereof is advantageous than that of the existing means for treating TTP, and can remedy the deficiency of the prior art.

The experiment results showed that, after treatment with antiplatelet thrombolysin, the platelet aggregation inhibition rate in the sample of Platelet Rich Plasma (PRP) in TTP patients was significantly improved compared with that of the control group. These results suggest that antiplatelet thrombolysin has a significant inhibitory effect on platelet aggregation in TTP patients, an effect on prevention of thrombosis and thrombocytopenia, and it has a good prospect of clinical application.

### DESCRIPTION OF FIGURES

FIG. 1 shows the inhibition effect on the antiplatelet thrombolysin in different concentrations on normal human plasma Ristocetin induced platelet aggregation. The green curve represents the platelet aggregation and time curve in blank control group; the purple curve represents the platelet aggregation and time curve after adding 1.5 ug/mL of antiplatelet thrombolysin; the blue curve represents platelet aggregation and time curve after adding 1.8 ug/mL of antiplatelet thrombolysin; and the black curve represents the platelet aggregation and time curve after adding 2.1 ug/mL of antiplatelet thrombolysin.

FIG. 2 shows the effect of antiplatelet thrombolysin on the activity of plasma ADAMTS13 in normal humans and TTP patients.

FIG. 3 shows the effect of anti ADAMTS13 antibody 3H9 on the concentration and activities of ADAMTS13 antigen in different groups, used for partially reflecting the establishment of TTP model. FIG. 3A shows after 3H9 injection, the ADAMTS13 enzymatic activity curves changed over time in control group (the legend is a solid circle), prevention group (the legend is hollow block) and treatment group (legend is solid triangle), FIG. 3B shows after 3H9 injection, the ADAMTS13 antigen concentration curves changed over time in the control group (the legend is a solid circle), prevention group (the legend is hollow block) and treatment group (legend is solid triangle).

FIG. 4 shows the prevention and treatment effects of antiplatelet thrombolysin on each index of thrombocytopenia and schistocyte hemolytic anemia. FIG. 4A shows the curves in platelet count changed over time in the control group (the legend is a solid circle), prevention group (the legend is hollow block) and treatment group (legend is solid triangle); FIG. 4B shows the haptoglobin concentration curves changed over time in the control group (the legend is a solid circle), prevention group (the legend is hollow block) and treatment group (legend is solid triangle); FIG. 4C shows the schistocyte count curve changed over time in the control group (the legend is a solid circle), prevention group (the legend is hollow block) and treatment group (legend is solid triangle); and FIG. 4D shows the lactate dehydrogenase level curves changed over time in the control group (the legend is a solid circle), prevention group (the legend is hollow block) and treatment group (legend is solid triangle).

### MODE OF CARRYING OUT THE INVENTION

The invention provides use of antiplatelet thrombolysin in the preparation of a medicament for the treatment of thrombotic thrombocytopenic purpura. The invention can be carried out by those skilled in the art through reading the disclosure herein with appropriate modification of process and parameters. In particular, all similar replacements and modifications are obvious for those skilled in the art, which are deemed to be within the scope of the invention. The method and application of the invention has been described through preferred examples. Those skilled in the relevant field can make change or appropriate modification and combination to the methods and uses described herein without departing from the content, spirit and scope of the invention, so as to achieve and apply the technology of the invention.

The reagents of the invention are all commercial products, and can all be purchased from the market. The following contents will further illustrate the invention in combination with examples:

### EXAMPLE 1

### The Inhibitory Effect of Antiplatelet Thrombolysin on Platelet Aggregation Induced by Ristocetin in Normal Humans and TTP Patients In Vitro

1. Sample Collection

3 cases of normal blood samples were collected from healthy blood donors. 3 TTP patients were selected from emergency TTP patients. The three patients all had typical clinical “TTP triad”, which ADAMTS13 activity in Laboratory testing was lower than 10%. The above samples were all from peripheral venous blood with trisodium citrate anticoagulant.

2. Experimental Method

(1) Inhibition test of platelet aggregation (Optical heterometry method): Whole blood containing 3.2% sodium citrate anticoagulant was taken and centrifuged at 800 PRM for 10 min to prepare PRP. The rest of the blood was centrifuged at 3000 PRM for 10 min to prepare platelet poor plasma (PPP). The PRP platelet concentration was adjusted to 300×109/L with PPP. The change of transmittance in 5 minutes was observed by adding ADP to PRP to a final concentration of 10 umol/L, after the PPP was adjusted to zero. The maximum transmittance was regarded as the maximum aggregation rate of PRP (PAGm).

$$Inhibition rate of platelet aggregation = \frac{PAGm without Thrombolysin - PAGm with Thrombolysin}{PAGm without Thrombolysin} \times 100 \%$$

(2) The test of plasma ADAMTS13 activity (Chemical fluorescence FRET method)

The plasma to be tested was mixed evenly with fluorescence buffer (5 mmol/L Bis-Tris, 25 mmol/L CaCl2, 0.005% Tween 20, pH 6) at a concentration of 1:25, meanwhile, normal human plasma was used as a standard sample, which was diluted with the above buffer in a ration of 1:12.5, 1:25, 1:50, 1:100, 1:200 and 1:400, as a standard curve. The mixed plasma of normal people in different concentrations and the samples to be tested were put into a 96-well half-skirt plate with 50 ul per well, then a certain concentration (2 umol/L) fluorescence labeled substrate FRETS-VWF73 with 50 ul per well was added into above 96 well plate for enzyme reaction with plasma to be tested. After 5 minutes, the reaction was detected in the microplate. reader The reaction temperature was set at 4° C., the fluorescence excitation/emission wavelength was 485/530 nm, and the frequency was set at one read every 2 minutes, the readings were terminated after 60 minutes. The whole process did not exceed 2 hours. The data of each well at each time point were curve-fitted to obtain the sample reaction curve equation. The slope of the test sample was put into the above log linear regression equation, with ADAMTS13 activity of 1:25 concentration normal mixed plasma as 100%, by comparing withwhich ADAMTS13 activity of the sample to be measured can be obtained.

3. Experimental Result

(1) Inhibition Test of Platelet Aggregation:

When the final concentration of the antiplatelet thrombolysin reached 1.8 ug/mL, the inhibition rate on the normal human plasma Ristocetin induced platelet aggregationcould be up to 93%. From the features of the aggregation curve and dose-effect relationship, antiplatelet thrombolysin mainly inhibited “the first phase” of platelet aggregation, that is, inhibits platelet activation and further aggregation by blocking the “outside-in” signaling pathway. The effect of inhibiting aggregation was very effective (FIG. 1).

Platelet aggregation induced by plasma Ristocetin in TTP patients was inhibited by antiplatelet thrombolysin. It was found from the results of the three TTP samples, 1.8 ug/mL concentration of antiplatelet thrombolysin was equally effective on inhibiting platelet aggregation in TTP patients (see table below). The following table shows the inhibitory effect of antiplatelet thrombolysin (1.8 ug/mL) on platelet aggregation in different populations.

| | Sample | | | | | |
|---|---|---|---|---|---|---|
| | Normal | | | TTP | | |
| | 1# | 2# | 3# | 1# | 2# | 3# |
| Maximum inhibition | 93% | 97% | 96% | 91% | 95% | 97% |
| rate | | | | | | |

| | | |
|---|---|---|
| Average | 95.3% | 94.3% |

(2) The Test of Plasma ADAMTS13 Activity

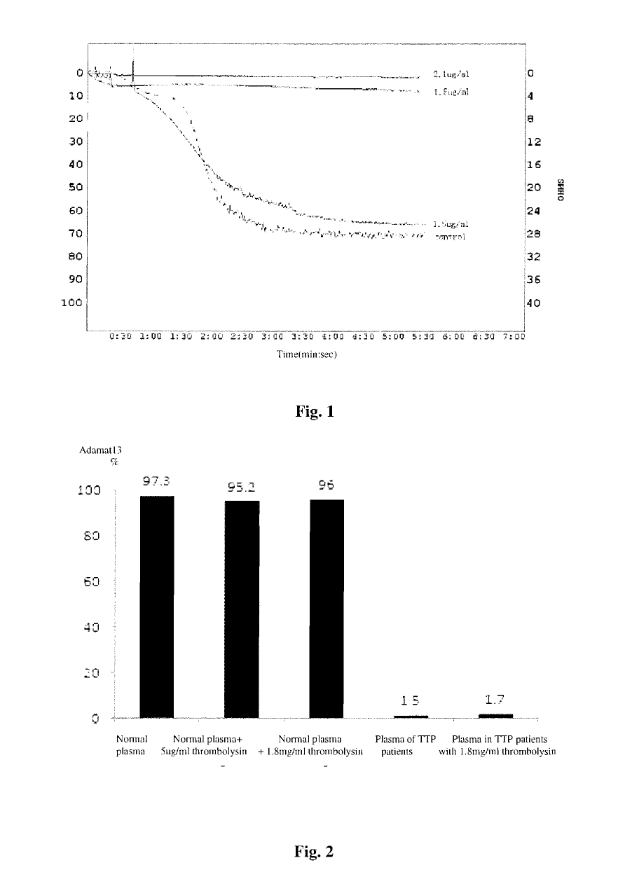

Antiplatelet thrombolysin of two different concentrations (5 ug/mL, 1.8 ug/mL) had no significant effect on plasma ADAMTS13 activity of normal humans or TTP patients (FIG. 2).

4. Conclusion

(1) The antiplatelet thrombolysin had obvious inhibition on plasma Ristocetin induced plate aggregation in normal humans or TTP patients. The batch (20130603) product with 1.8 ug/mL (final concentration) could achieve the maximum inhibition rate of more than 90%, and there was no difference in inhibition on platelet aggregation in TTP patients and normal humans.

(2) The normal therapeutic concentration of antiplatelet thrombolysin had no significant effect on the ADAMTS13 activity in normal human plasma, showing that it did not inhibit the behavior of the normal ADAMTS13 cleaving vWF.

### EXAMPLE 2

### Effectiveness Experiment for Antiplatelet Thrombolysin on Acquired TTP in Baboon Model

1. Experimental Design and Method

The experiment was divided into three groups, with 4 animals in each group. The first group was used as positive control. From day 0 of the experiment, the animals were injected with anti ADAMTS13 antibody 3H9 at 600 ug/kg every 48 hours through femoral vein to establish the TTP model, until day 9 of the experiment, i.e., the end of the experiment The second group was the preventive group, and the same dose of 3H9 was injected on day 1 and day 3, the 0.3 mg/kg antiplatelet thrombolysin was subcutaneously injected on day 1 and day 5 to observe its preventive effect on TTP. The third group was the treatment group, starting from day 0 of the trial, which was injected with anti DAMTS13 antibody 3H9 at 600 ug/kg every 48 hours through femoral vein, until day 9, i.e., the end of the experiment, to establish TTP model. Among them, from day 5 to day 11, 0.3 mg/kg of antiplatelet thrombolysin was injected subcutaneously every day to observe its therapeutic effect on TIP.

Blood samples were taken every day and hematologic parameters were measured, which include: whole blood cell count, lactate dehydrogenase level, haptoglobin level, schistocyte count and the like. The ADAMTS13 antigen concentration and activity parameters were determined by specific ELISA test.

2. Experimental Results

(1) Establishment of TTP Model:

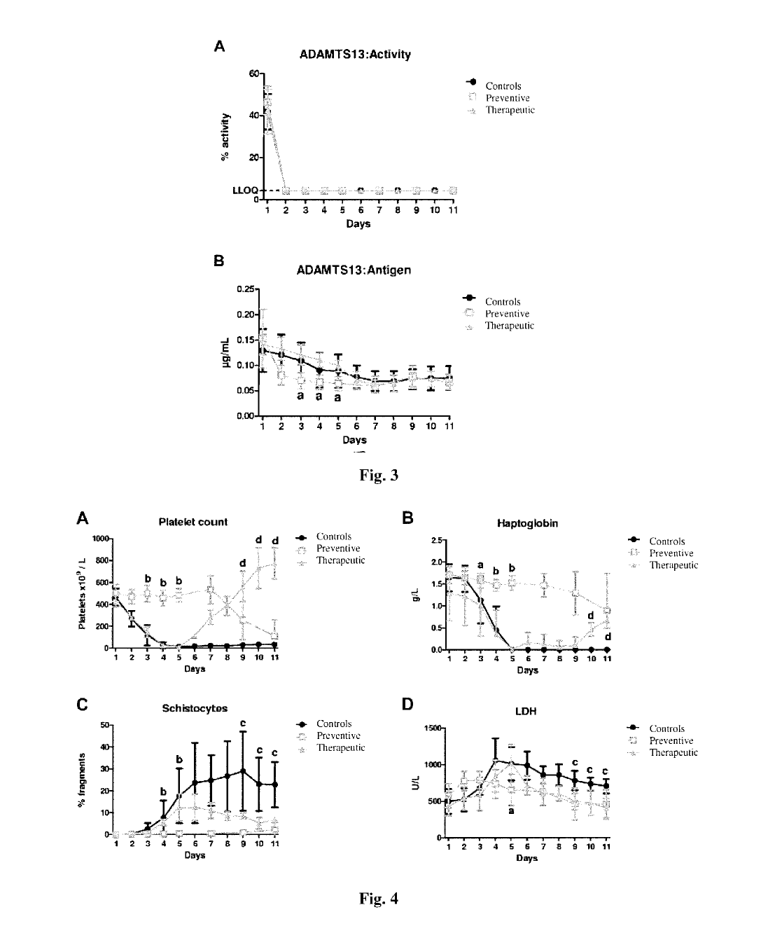

The injection of anti ADAMTS13 antibody 3H9 every 48 hours resulted in significant decrease of ADAMTS13 enzyme activity, which also causes the decrease of the concentration of ADAMTS13 antigen (FIG. 3). It directly resulted in thrombocytopenia and hemolytic anemia in animals, platelet count and haptoglobin count were reduced, schistocyte count and lactic dehydrogenase levels were increased (FIG. 4). These are all typical symptoms of TTP, and are also indicators of the success of the model establishment.

(2) Effectiveness of Thrombolysin in Prevention Group:

Compared with the control group, in the prevention group, antiplatelet thrombolysin prevented the reduction of platelet count, and inhibited the occurrence of symptoms including schistocyte hemolytic anemia and abnormal levels of lactate dehydrogenase. No reduction of red blood cell count and hemoglobin concentration were observed in this group of animals (FIG. 4).

(3) Effetiveness of Thrombolysin in the Treatment Group:

In the treatment group, TTP symptoms were observed on t day 5 of the experiment, all indexes were consistent with those of the control group, indicating that the model was well established. Platelet count increased significantly after antiplatelet thrombolysin injection on day 5, and recovered the initial level onday 9, and increased to 180% of the initial level on day 11. schistocyte hemolytic anemia was effectively treated after day 5, the schistocyte count also decreased significantly, but the haptoglobin concentration tended to normalize from day 10 (FIG. 4).

3. Conclusion

Experiments have demonstrated that antiplatelet thrombolysin can prevent and treat the major symptoms of acquired TTP in baboon model, restore platelet, red blood cell and hemoglobin levels, at the same time, reduce the level of lactate dehydrogenase, effectively inhibit thrombocytopenia and schistocyte hemolytic anemia so as to achieve the purpose of the treatment of TTP.

The use of antiplatelet thrombolysin in the preparation of a medicament for the treatment of thrombotic thrombocytopenic purpura provided by the invention has been described through examples. Those skilled in the relevant field can make change or appropriate modification and combination to the use of antiplatelet thrombolysin in the preparation of a medicament for the treatment of thrombotic thrombocytopenic purpura described herein without departing from the contents, spirit and scope of the invention, so as to achieve the technology of the invention. In particular, all similar replacements and modifications are obvious for those skilled in the art, which are deemed to be included in the spirit, scope and contents of the invention.

1. (canceled)

2. (canceled)

3. (canceled)

4. (canceled)

5. (canceled)

6. (canceled)

7. (canceled)

8. (canceled)

9. A method of treating a mammal suffering from thrombotic thrombocytopenic purpura comprising:
administering to a mammal having thrombotic thrombocytopenic purpura a therapeutically effective amount of an antiplatelet thrombolysin comprising two polypeptide chains, an alpha chain having an amino acid sequence of SEQ ID NO: 1 and a beta chain having an amino acid sequence of SEQ ID NO.2.

10. The method of treating a mammal suffering from thrombotic thrombocytopenic purpura according to claim 9, wherein said thrombotic thrombocytopenic purpura is congenital or acquired thrombotic thrombocytopenic purpura.

11. The method of treating a mammal suffering from thrombotic thrombocytopenic purpura according to claim 9, wherein said antiplatelet thrombolysin is a pharmaceutical composition made by a chemical or biological process.

12. The method of treating a mammal suffering from thrombotic thrombocytopenic purpura according to claim 9, wherein said antiplatelet thrombolysin is a pharmaceutical composition further comprising a pharmaceutically acceptable excipient.

13. The method of treating a mammal suffering from thrombotic thrombocytopenic purpura according to claim 9, wherein said antiplatelet thrombolysin is an oral preparation or an injection.

14. The method of treating a mammal suffering from thrombotic thrombocytopenic purpura according to claim 13, wherein said oral preparation is a tablet, a capsule, a pill, a granule, a drop pill, a microcapsule or a micropellet.

15. The method of treating a mammal suffering from thrombotic thrombocytopenic purpura according to claim 13, wherein a mass-volume concentration of the antiplatelet thrombolysin in the oral preparation or injection is 1.8-10 μg/mL.

16. The method of treating a mammal suffering from thrombotic thrombocytopenic purpura according to claim 13, wherein a dose of the antiplatelet thrombolysin in the oral preparation or injection is 0.03-3 mg/kg.


SEQUENCE LISTING (2 sequences)

SEQ ID NO: 1 - protein, 130 aa, Artificial Sequence
  Description of Artificial Sequence  Artificial polypeptide
DVDCLPGWSAYDQSCYRVFKLLKTWDDAEKFCTERPKGGHLVSIESAGERDFVAQLVSENKQTDNVWLGLKIQSKGQQCSTEWTDGSSVSYENFSEYQSKKCFVEKNTGFRTWLNLNCGSEYAFVCKSPP

SEQ ID NO: 2 - protein, 125 aa, Artificial Sequence
  Description of Artificial Sequence  Artificial polypeptide
GFCCPLRWSSYEGHCYLVVKEKKTWDDAEKFCTEQRKGGHLVSVHSREEADFLVHLAYPILDLSLIWMGLSNMWNDCKREWSDGTKLDFKAWAKTSDCLIGKTDDNQWLNMDCSKKHYFVCKFKL